(19) United States
(12) Patent Application Publication
(10) Pub. No.: US 2005/0228469 A1
(43) Pub. Date: Oct. 13, 2005

(54) Electrode and conductor interconnect and method therefor

(75) Inventors: Paul E. Zarembo, Vadnais Heights, MN (US); Lyle A. Bye, Lino Lakes, MN (US); Daniel J. Cooke, Roseville, MN (US); Christina Repasky, Maplewood, MN (US)

Correspondence Address: SCHWEGMAN, LUNDBERG, WOESSNER & KLUTH, P.A., P.O. BOX 2938, MINNEAPOLIS, MN, 55402-0938, US

(73) Assignee: Cardiac Pacemakers, Inc.

(21) Appl. No.: 10/822,604

(22) Filed: Apr. 12, 2004

(51) Int. Cl. A61N 1/05

(52) U.S. Cl. 607/122

(57) ABSTRACT

A lead assembly is provided that includes an outer insulative body, at least one conductor disposed within the outer insulative body, and an inner electrode coupled with the at least one conductor. The lead assembly further includes an outer electrode disposed over the inner electrode, where the outer electrode coupled with at least a portion of the inner electrode outer surface, for example by laser welding or magnetic swaging.

### TECHNICAL FIELD

Leads for conducting electrical signals to and from the heart, and more particularly, leads having an interconnect for one or more electrodes and the conductor.

### TECHNICAL BACKGROUND

Pacemaker leads represent the electrical link between the pulse generator and the heart tissue, which is to be excited and/or sensed. These pacemaker leads include single or multiconductors that are connected to an electrode in an electrode assembly at an intermediate portion or distal end of a pacing lead. A connector is included at the proximal end to form the electrical connection with the pacemaker.

To implant the lead within the patient, the lead is often fed intravenously toward the heart. The lead may be implanted within or travel through complex or tortuous vasculature. The lead may also need to travel through vasculature having increasingly smaller diameters. As leads are designed smaller, the electrodes associated with the leads are also designed smaller, and need to be treated more carefully during manufacturing, while providing a robust design.

Accordingly, what is needed is a lead having a lead body with reduced outer diameter that maintains a design that can withstand travel through the tortuous vasculature, as well as manufacturing processes without failure.

### SUMMARY

A lead assembly is provided that includes an outer insulative body, at least one conductor disposed within the outer insulative body, and an inner electrode coupled with the at least one conductor. The lead assembly further includes an outer electrode disposed over the inner electrode, where the outer electrode coupled with at least a portion of the inner electrode outer surface.

Several options for the lead assembly are as follows. For instance, in one option, the inner electrode includes an outer surface that has a stepped portion having a ledge, and the conductor is disposed on the ledge. In another option, a portion of the conductor is disposed between the inner electrode outer surface and the outer electrode, and further the conductor extends to a distal end, and optionally, the distal end is disposed between the inner electrode outer surface and the outer electrode. In another option, at least one of the inner electrode outer surface or the outer electrode inner surface include interlocking features.

In another embodiment, the lead assembly includes an outer insulative body, at least one conductor disposed within the outer insulative body, and an inner electrode coupled with the at least one conductor. An outer electrode is disposed over the inner electrode, where the outer electrode has a treated outer surface. The lead assembly further includes means for electrically and mechanically coupling the outer electrode with the inner electrode without substantially damaging the treated outer surface of the outer electrode.

Several options for the lead assembly are as follows. For example, in one option, a portion of the inner electrode and the outer electrode are formed of shape memory material. In another option, a portion of the conductor is disposed between the outer electrode and the inner electrode.

A method is further provided where the method includes coupling a conductor with an inner electrode, inspecting the coupling between the inner electrode and the conductor, coupling the inner electrode with the outer electrode after the coupling between the inner electrode and the conductor is inspected, and disposing insulative tubing over the conductor and inner electrode.

Several options for the method are as follows. For example, in one option, coupling the conductor with the inner electrode includes laser welding the conductor to the inner electrode, for example a central section of the inner electrode. In another option, coupling the conductor with the inner electrode includes coupling the conductor with a ledge on an outer surface of the inner electrode. The method further optionally includes abutting a distal end of the conductor with a stepped surface of the inner electrode.

In another embodiment, a method includes coupling a conductor with an inner electrode, disposing an outer electrode over the inner electrode, coupling at least one of the inner electrode or the conductor with the outer electrode without directly contacting the outer surface of the outer electrode, disposing insulative tubing over at least a portion of the conductor, and the inner electrode.

Several options exist for the above discussed methods. For example, in one option, coupling at least one of the inner electrode or the conductor with the outer electrode includes magnetic flux swaging the outer electrode. In another option, coupling at least one of the inner electrode or the conductor with the outer electrode includes resistance welding the outer electrode.

These and other embodiments, aspects, advantages, and features will be set forth in part in the description which follows, and in part will become apparent to those skilled in the art by reference to the following description and referenced drawings or by practice thereof. The aspects, advantages, and features are realized and attained by means of the instrumentalities, procedures, and combinations particularly pointed out in the appended claims and their equivalents.

### BRIEF DESCRIPTION OF THE DRAWINGS

FIG. 1 is a diagram illustrating a lead system constructed in accordance with one embodiment.

FIG. 2 is a cross-sectional view of a portion of a lead constructed in accordance with one embodiment.

FIG. 3 is a detailed view of an electrode assembly constructed in accordance with one embodiment.

FIG. 4 is a cross-sectional view of an inner electrode constructed in accordance with one embodiment.

FIG. 5A is an end view of an outer electrode constructed in accordance with one embodiment.

FIG. 5B is a cross-sectional view of an outer electrode constructed in accordance with one embodiment.

FIG. 6A is a cross-section view of an electrode assembly constructed in accordance with one embodiment.

FIG. 6B is a cross-section view of an electrode assembly constructed in accordance with one embodiment.

FIG. 6C is a cross-section view of an electrode assembly constructed in accordance with one embodiment.

FIG. 6D is a cross-section view of an electrode assembly constructed in accordance with one embodiment.

FIG. 6E is a cross-section view of an electrode assembly constructed in accordance with one embodiment.

FIG. 6F is a perspective view of an electrode assembly constructed in accordance with one embodiment.

FIG. 7 is a cross-section view of an electrode assembly constructed in accordance with one embodiment.

### DESCRIPTION OF THE EMBODIMENTS

In the following detailed description, reference is made to the accompanying drawings, which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the spirit and scope of the present invention. Therefore, the following detailed description is not to be taken in a limiting sense, and the scope is defined by the appended claims.

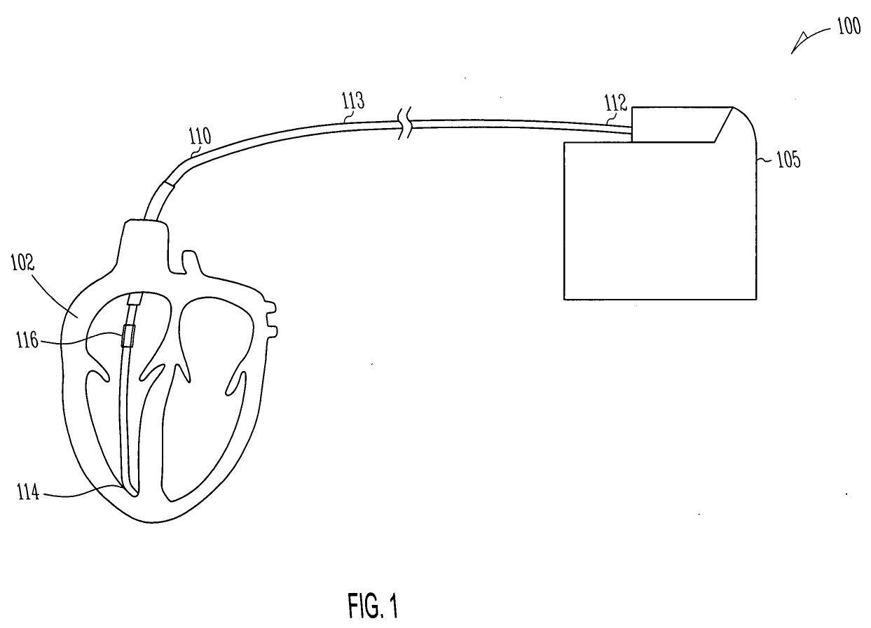

A lead assembly 110 and lead system 100 are illustrated in FIG. 1. FIG. 1 is a diagram of a system 100 for delivering and/or receiving electrical pulses or signals to stimulate, shock, and/or sense the heart 102. The system 100 includes a pulse generator 105 and a lead 110. The pulse generator 105 includes a source of power as well as an electronic circuitry portion. The pulse generator 105, in one option, is a battery-powered device which generates a series of timed electrical discharges or pulses. The pulse generator 105 is generally implanted into a subcutaneous pocket made in the wall of the chest. Alternatively, the pulse generator 105 is placed in a subcutaneous pocket made in the abdomen, or in other locations. It should be noted that while the lead assembly 110 is illustrated for use with a heart, the lead assembly 110 is suitable for other forms of stimulation as well. For example, the lead assembly 110 can be used for neuro stimulation.

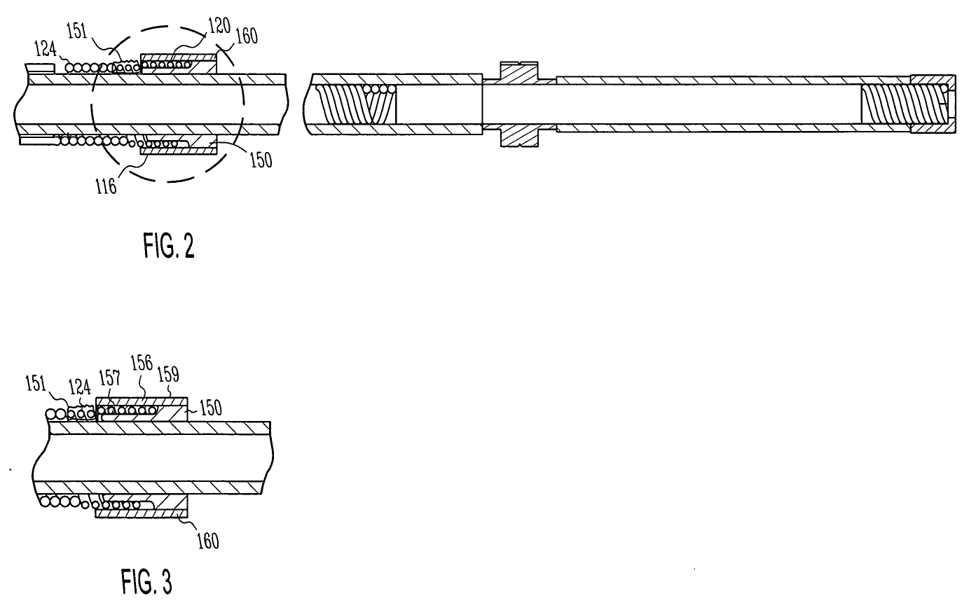

The lead assembly 110 includes a lead body 113 which extends from a proximal end 112, where it is coupled with the pulse generator 105, as further discussed below. The lead assembly 110 extends to a distal end 114, which is coupled with a portion of a heart 102, when implanted. The distal end 114 of the lead assembly 110 includes at least one electrode assembly 116 which electrically couples the lead assembly 110 with the heart 102. At least one electrical conductor 120 (FIG. 2) is disposed within the lead assembly 110 and extends, in one option, from the proximal end 112 to the distal end 114 of the lead assembly 110. The at least one electrical conductor 120 electrically couples the electrode assembly 116 with the proximal end 112 of the lead assembly 110. The electrical conductors carry electrical current and pulses between the pulse generator 105 and the electrode assembly 116, and to and from the heart 102.

Referring to FIG. 2, the lead assembly 110 includes at least one conductor 120, for example a coiled conductor or a cable conductor. In one option, a plurality of conductors is disposed therein. For example, a plurality of cable conductors is disposed within the lead body 110. In another option, a plurality of coiled conductors 124 is disposed within the lead body 110. The coiled conductors 124 include at least one filar, and optionally at least a portion of each individual filar is insulated. The at least one conductor extends from a proximal end to a distal end.

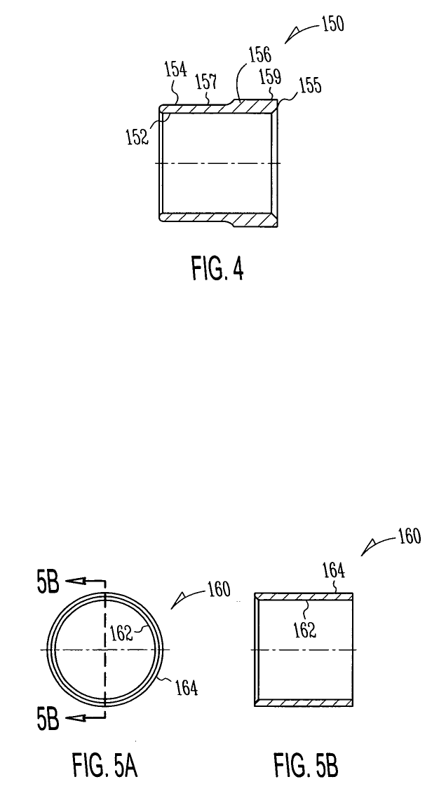

The lead assembly further includes the electrode assembly 116, that includes an inner electrode 150 and an outer electrode 160, where at least a portion of the inner electrode 150 is disposed within the outer electrode 160. The inner electrode 150, illustrated in greater detail in FIGS. 3 and 4 is defined in part by an inner surface 152, an outer surface 154, and end surfaces 155. In one option, the outer surface 154 of the inner electrode 150 includes a stepped portion 156. The stepped portion 156 allows for the inner electrode 150 to have two or more outer diameters. In one option, one of the outer diameters forms a ledge 157 such that the coil 124 is disposed on the ledge 157. The diameter of the ledge 157, in one option, has a larger outer diameter than the inner diameter of the coil 124, prior to the assembly of the two together. This allows for the coil 124 to more snugly grasp the outer diameter of the ledge 157. In one option, the end of the coil 124 rests adjacent to the larger outer diameter, and abutting the larger diameter. This allows for the coil conductor 124 to be positioned axially along the lead, for example when the distal end of the coil conductor 124 is disposed adjacent to abut the stepped portion 156.

The conductor 120, for example, the coil conductor 124, is coupled to the ledge 157, for example by welding such as laser welding. This allows for the coupling between the conductor 120 and the electrode assembly 116 (FIG. 1). The welding process can occur before the attachment of the outer electrode thereto, such that the coupling can be inspected visually, for example, before the outer electrode is attached. Furthermore, the welding or coupling process is a visible welding or coupling process, further ensuring an electrical connection between the conductor and the electrode assembly.

The stepped portion 156 of the inner electrode 150 includes a substantially smooth outer surface 159 to which the outer electrode 160 is coupled or welded. The outer surface 159 forms an outer continuous ring, for example, to which the outer electrode 160 can be welded, such as by laser welding or resistance welding. Alternatively, the distal ends of the inner and outer electrodes 150, 160 can be laser welded so as not to affect the outer electrode surface. This arrangement allows for the electrode assembly to be connected with the conductor without substantially interfering with the surface of the outer electrode, or without substantially damaging the outer surface of the outer electrode. In another option, adhesive 151 can be used to improve the integrity of the lead, for example, under axial load.

Referring to FIGS. 5A and 5B, the outer electrode 160 is, in one option, a ring electrode and is defined in part by an inner surface 162 and an outer surface 164. The outer surface 164 of the outer electrode 160, in one option, includes a coated or treated surface, for example a coating of IrOx or titanium oxide is coated on at least a portion of the outer electrode 160.

In one option the inner surface 162 of the outer electrode 160 is configured to couple directly with a portion of the inner electrode 150, for example, directly along the outer surface 159 of the inner electrode 150, as discussed above. In another option, a portion of the conductor 120, for example, the coiled conductor 124, is placed in between the outer electrode 160 and the inner electrode 150, where the outer and inner electrodes 160, 150 are coupled with the coiled conductor 124, for example by magnetic flux swaging, resistance welding, or laser welding. The magnetic flux swaging may be assisted by using a tubular component outside of the outer electrode that is more conducive to magnetic flux swaging to radially compress the outer electrode. For instance, a supplemental outer ring can be placed over the outer electrode with protective tubing, where the supplemental outer ring is more conductive. In another option, a barrier or protective material, such as heat shrink tubing, is disposed between the outer electrode and the supplemental outer ring to minimize transfer of metal or damage to coatings. Ridges may further be placed on the internal diameter of the more conductive tubular component to localize contact and to minimize damage.

One example of the supplemental outer ring includes a ring with high electrical conductivity and movable by magnetic flux, for example, a copper ring. The supplemental outer ring can be magnetically swaged to compress the outer electrode 160 around the conductor 120 and the inner electrode 150. After the coupling process, the supplemental outer ring and the protective tubing can be removed. The supplemental outer ring optionally includes ridges on the internal diameter to localize contact.

In another option, shape memory metal can be used to couple the outer electrode with the inner electrode. For example, the outer electrode is formed of shape memory material, such as nitinol, and the outer surface or outer portion of the outer electrode 160 contracts to couple the outer electrode 160 with the conductor 120 and/or the inner electrode 150. In another option, the inner electrode and the outer electrode 160 is formed of shape memory material, such as nitinol. The outer electrode 160 contracts and the inner electrode expands to couple the components together. In yet another option, the inner electrode 150 is formed of shape memory material, such as nitinol, and expands to couple the conductor and the inner and outer electrodes together.

In one embodiment, the conductor 120 is sandwiched between the inner electrode 150 and the outer electrode 160, and the conductor 120, the inner electrode 150 and the outer electrode 160 are mechanically coupled together without substantially damaging or disturbing the outer surface of the outer electrode 160. In another option, the conductor 120, the inner electrode, and the outer electrode 160 are coupled together at substantially the same time. For example, outer electrode 160 is magnetically formed or swaged inward toward the coil and the inner electrode. Alternatively, magnetic pulse welding can be used. In one option, a tube or mandrel is disposed within to assist in the mechanical attachment. It should be noted that the assembly can be formed without the inner electrode, for example, the outer electrode and the coil can be coupled together by magnetic swaging. Advantageously, there is no heat affected zone, thereby assisting in preventing damage to the coil at the attachment point.

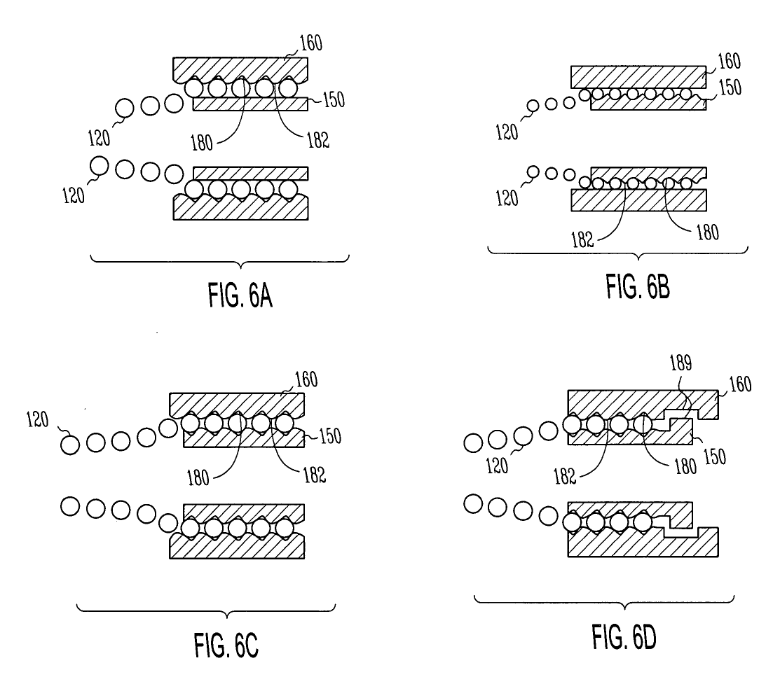

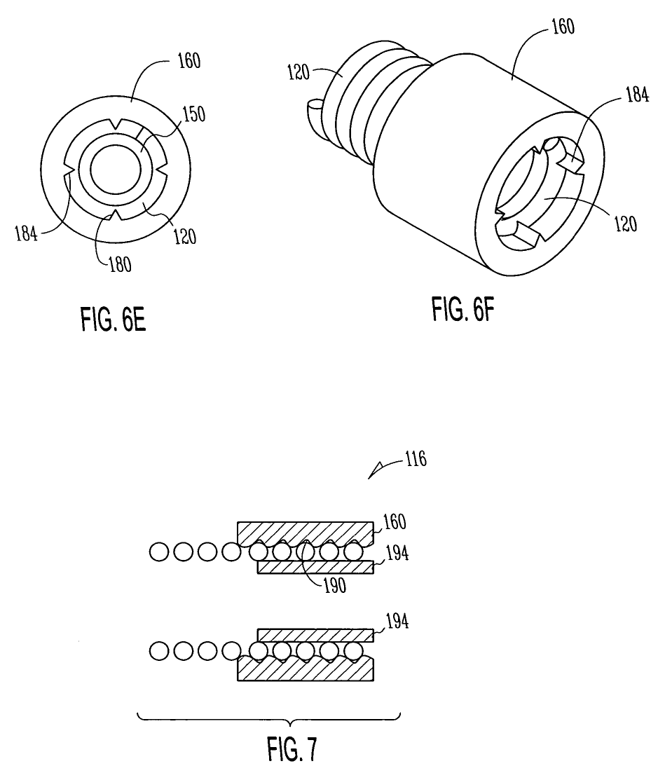

In one option, the inner electrode 150 and/or the outer electrode 160 include features 180 that remove or disrupt a portion of insulation from the conductor 120, for example by splitting, cutting, or interrupting the insulation, to allow for an electrical connection to the conductive filar. In one option, the disruption to the insulation occurs during the interconnection process, i.e. the magnetic welding. For example, as illustrated in FIGS. 6A-6E, the insulation disruption features 180 include threads 182 disposed on an inner surface of the outer electrode 160, an outer surface of the inner electrode 150, or both. In another option, a sharp feature 184 can be disposed along a portion of the inner or outer electrodes 150, 160, for example, as illustrated in FIG. 6E.

Other suitable features include having an outer surface of the inner electrode different than the inner surface of the outer electrode, for example, a square outer surface and a cylindrical inner surface. In addition, features can be added to the inner and outer electrodes where they contact the coil to improve axial strength and electrical conductivity, such as, but not limited to, threads, ridges, grooves, bumps, or holes. The features further optionally include sharp edges allowing for the features to create a gas tight seal, or to cut through oxide layers or insulation on the coil as the outer ring is coupled, for example, by magnetically swaging the outer electrode.

As the outer and inner electrodes are assembled together, the features 180 cut into, interrupt, or split the insulation away from the conductive portion of the conductor 120, allowing the conductor 120 to be electrically coupled with the outer and/or inner electrodes 160,150. In another option, the features 180 or features 189 form an interconnect between the inner electrode 150 and the outer electrode 160.

A method is further provided where the method includes coupling a conductor with an inner electrode, and an outer electrode, for example for the components and various embodiments discussed above and/or illustrated in the drawings. The coupling of the inner electrode and the conductor allows for an inspecting of the coupling between the inner electrode and the conductor, visually. Furthermore, the coupling of the conductor with the inner electrode can be done visually, for example by welding or laser welding, such that there is no blind welding. The method further includes coupling the inner electrode with the outer electrode, for example by laser welding, after the coupling between the inner electrode and the conductor is inspected, and disposing insulative tubing over the conductor and inner electrode, where the tubing is disposed, in one option, before the outer electrode is coupled with the inner electrode. This minimizes the potential damage or handling of the outer electrode or damage to an outer coating on the outer electrode.

Several options for the method are as follows. For example, in one option, coupling the conductor with the inner electrode includes laser welding the conductor to the inner electrode, for example a central section of the inner electrode. In another option, coupling the conductor with the inner electrode includes coupling the conductor with a ledge on an outer surface of the inner electrode, as discussed above. The method further optionally includes abutting a distal end of the conductor with a stepped surface of the inner electrode. This allows for the loose filar ends to be held in place prior to stringing of the outer insulation, or other components, to protect the coil and the components to be strung thereover. Furthermore, subassemblies of an inner coil and an outer coil assembly can be strung over each other, aiding in the flexibility of manufacturing, throughput, and yields. For instance, failing subassemblies can be scrapped, as opposed to the entire unit.

In another embodiment, a method includes coupling a conductor with an inner electrode, for example by disposing the conductor on a portion of the inner electrode. Alternatively, the conductor can be welded or otherwise coupled with the inner electrode. An outer electrode is disposed over the inner electrode, and the method further includes coupling at least one of the inner electrode or the conductor with the outer electrode without directly contacting the outer surface of the outer electrode, for example, as illustrated in FIG. 7. The weld joint 190 of the electrode assembly 116 is formed for example by magnetic pulse, such as by applying a magnetic swage force the outer electrode toward the inner electrode and the conductor. Alternatively, the outer electrode, the inner electrode and the conductor can be coupled together with resistance welding.

Inclusion of the inner electrode ring is optional, and the outer electrode can be coupled with the conductor by using magnetic pulse welding, where the outer electrode does not need to be directly contacted during the coupling process. In one option, a mandrel 194 is used during the welding process, and removed after the weld, thereby avoiding mechanical clamping of the outer surface of the outer electrode 160, for example, as required in standard mechanical swaging process. Since the outer surface of the outer electrode is not directly contacted, the outer coating, for example, of IrOx, is not affected by the attachment process. It should be noted that the magnetic welding process can be used for forming the interconnects between other components within the lead assembly as well, including for example, asymmetric components. If an adjacent aperture or lumen is present, a mandrel or low electrical conductivity tube can be used to prevent collapse of the adjacent lumen.

The insulative tubing for the lead can be disposed over the electrode assembly formed by the inner and outer electrodes, and the conductor. Alternatively, the insulative tubing can be disposed over the inner electrode and the conductor, allowing for the outer electrode to be disposed and coupled with the inner electrode later in the manufacturing process. This aids in preventing substantially damage to the outer electrode, such as a coating of the outer electrode. Other ways in preventing substantial damage to the outer electrode include coupling the outer electrode with the inner electrode in a way to prevent mechanical clamping directly to the outer surface of the outer electrode, for example, by magnetically swaging the outer electrode to the inner electrode.

Several options exist for the above discussed methods. For example, in one option, coupling at least one of the inner electrode or the conductor with the outer electrode includes magnetic flux swaging the outer electrode. In another option, coupling at least one of the inner electrode or the conductor with the outer electrode includes resistance welding the outer electrode. Other options include incorporating the various options discussed above, such as, but not limited to, including insulation disruption features on the inner and outer electrodes, and disrupting the insulation on the conductor when the components are assembled or coupled together, for example, by magnetically swaging the components. For example, the insulation can be disrupted with features, such as ridges on an internal portion of the outer electrodes, and/or an external portion of the inner electrode. Alternatively, or in addition to that above, the insulation can be disrupted by crushing through the insulation, for example, with an appropriate amount of force, or for example, with asymmetric components. In another option, the inner and outer electrodes includes interlocking features disposed between.

Advantageously, the current design assists in eliminating the need for manufacturing operations that may cause lower yields. For example, the design allows for ends of conductors such as coils to be covered, preventing sharp filar issues, for example, during the stringing process of the insulative tubing. Alternative designs further assist in the elimination of manual stripping of the conductors prior to attachment of the electrode. The design allows the outer insulation to be placed on the coil after the inner electrode is coupled with the coil, protecting the coil from potential damage during stringing of the outer insulation, and further minimizing the handling of the outer electrode during the manufacturing process. Since the subassembly can be inspected before the final assembled product, defective parts can be scrapped earlier in the manufacturing process, resulting in a cost savings. The design also allows for shorter electrodes, which are easier to manipulate through the vasculature of a patient.

It is to be understood that the above description is intended to be illustrative, and not restrictive. Although the use of the implantable device has been described for use as a lead in, for example, a cardiac stimulation system, the implantable device could as well be applied to other types of body stimulating systems. Furthermore, it should be noted that the embodiments, and various options described above and illustrated in the drawings, may be selectively combined to form additional embodiments. Many other embodiments will be apparent to those of skill in the art upon reviewing the above description. The scope should, therefore, be determined with reference to the appended claims, along with the full scope of equivalents to which such claims are entitled.

1. A lead assembly comprising:
an outer insulative body;
at least one conductor disposed within the outer insulative body, the at least one conductor extending from a conductor proximal end to a conductor distal end;
an inner electrode coupled with the at least one conductor, the at least one conductor welded to the inner electrode, the inner electrode defined in part by an inner electrode inner surface, an inner electrode outer surface and inner electrode end surfaces; and
an outer electrode disposed over the inner electrode, the outer electrode coupled with at least a portion of the inner electrode outer surface.

2. The lead assembly as recited in claim 1, wherein the inner electrode outer surface includes a stepped portion having a ledge, and the conductor is disposed on the ledge.

3. The lead assembly as recited in claim 1, wherein the inner electrode and the outer electrode are welded together.

4. The lead assembly as recited in claim 1, wherein a portion of the conductor is disposed between the inner electrode outer surface and the outer electrode.

5. The lead assembly as recited in claim 4, wherein the at least one conductor extends to a distal end, and the distal end is disposed between the inner electrode outer surface and the outer electrode.

6. The lead assembly as recited in claim 1, wherein at least one of the inner electrode outer surface or the outer electrode inner surface include insulation disruption features.

7. A lead assembly comprising:
an outer insulative body;
at least one conductor disposed within the outer insulative body, the at least one conductor extending from a conductor proximal end to a conductor distal end;
an inner electrode coupled with the at least one conductor, the inner electrode defined in part by an inner electrode inner surface, an inner electrode outer surface and inner electrode end surfaces;
an outer electrode disposed over the inner electrode, the outer electrode having a treated outer surface; and
means for electrically and mechanically coupling the outer electrode with the inner electrode without substantially damaging the treated outer surface of the outer electrode.

8. The lead assembly as recited in claim 7, wherein the means for electrically and mechanically coupling the outer electrode with the inner electrode includes a laser welded coupling formed between the inner electrode and the outer electrode.

9. The lead assembly as recited in claim 7, wherein the means for electrically and mechanically coupling the outer electrode with the inner electrode includes a magnetic swage coupling.

10. The lead assembly as recited in claim 7, wherein at least a portion of at least one of the inner electrode and the outer electrode are formed of shape memory material.

11. The lead assembly as recited in claim 7, wherein the conductor is disposed between the outer electrode and the inner electrode, and the outer electrode, the conductor, and the inner electrode are coupled together at substantially the same time.

12. The lead assembly as recited in claim 7, further comprising means for disrupting insulation disposed between the inner and outer electrode.

13. A method comprising:
coupling a conductor with an inner electrode to form a conductor-inner electrode coupling,
inspecting the coupling between the inner electrode and the conductor;
coupling an outer electrode with the inner electrode after the inspecting the conductor-inner electrode coupling between the inner electrode and the conductor; and
disposing insulative tubing over the conductor.

14. The method as recited in claim 13, wherein coupling the conductor with the inner electrode includes laser welding the conductor to the inner electrode.

15. The method as recited in claim 13, wherein coupling the inner electrode with the outer electrode includes laser welding the outer electrode to a central section of the inner electrode.

16. The method as recited in claim 13, wherein coupling the conductor with the inner electrode includes coupling the conductor with a ledge on an outer surface of the inner electrode.

17. The method as recited in claim 13, wherein disposing insulative tubing over the conductor occurs prior to coupling the outer electrode with the inner electrode.

18. A method comprising:
disposing a conductor over at least a portion of an inner electrode;
disposing an outer electrode over the inner electrode and the conductor, the outer electrode having an outer surface;
coupling the inner electrode, the conductor, and the outer electrode without directly contacting the outer surface of the outer electrode; and
disposing insulative tubing over at least a portion of the conductor, and the inner electrode.

19. The method as recited in claim 18, wherein coupling at least one of the inner electrode or the conductor with the outer electrode includes magnetic flux swaging the outer electrode, the conductor and the inner electrode together.

20. The method as recited in claim 18, wherein coupling the inner electrode, the conductor, and the outer electrode together includes resistance welding the outer electrode, the conductor and the inner electrode.

21. The method as recited in claim 18, further comprising interlocking the outer electrode with the inner electrode with interlocking features disposed therebetween.

22. The method as recited in claim 18, wherein coupling the outer electrode, the inner electrode, and the conductor includes forming at least a portion of the outer electrode with shape memory material, and contracting the outer electrode.

23. The method as recited in claim 22, wherein contracting the outer electrode includes contracting an outer surface of the outer electrode.

24. The method as recited in claim 22, wherein contracting the outer electrode includes contracting an inner surface of the outer electrode.

25. The method as recited in claim 18, further comprising disrupting insulation of the conductor with features formed along a portion of the inner electrode or the outer electrode.
